(19) United States
(12) Patent Application Publication
(10) Pub. No.: US 2022/0133259 A1
(43) Pub. Date: May 5, 2022

(54) BLADDER MONITORING APPARATUS AND METHOD FOR CONTROLLING BLADDER MONITORING APPARATUS

(71) Applicant: DAEGU GYEONGBUK INSTITUTE OF SCIENCE AND TECHNOLOGY, Daegu (KR)

(72) Inventors: Jae Youn HWANG, Daegu (KR); Moon Hwan LEE, Gyeongsan-si (KR); Hah Min LEW, Seoul (KR)

(21) Appl. No.: 17/516,850

(22) Filed: Nov. 2, 2021

(30) Foreign Application Priority Data
Nov. 3, 2020 (KR) 10-2020-0145463

(51) Int. Cl. A61B 8/08; G06T 7/62; G06T 7/00; A61B 8/00; G16H 30/20; G16H 50/20

(52) U.S. Cl. CPC A61B 8/08; G06T 7/62; G06T 7/0012; A61B 8/5223; A61B 8/4416; A61B 8/4227; A61M 25/0017; G16H 30/20; G16H 50/20; G06T 2207/10132; G06T 2207/20081; G06T 2207/20084; A61B 8/4236

(57) ABSTRACT

Disclosed are a bladder monitoring apparatus which accurately determines a bladder status of a subject based on an ultrasonic image and a posture of the subject and a method for controlling the bladder monitoring apparatus. The bladder monitoring apparatus includes a processor, a memory configured to operably connected to the processor and store at least one code executed by the processor, and a transceiver configured to receive a reflection ultrasonic signal from a subject and a posture sensing signal obtained by sensing a posture of the subject based on a sensor, and the memory may store a code which is executed by the processor to cause the processor to determine the bladder status of the subject by applying the machine learning-based learning model to an ultrasonic image generated from the reflection ultrasonic signal and posture information generated based on the posture sensing signal.

### STATEMENT REGARDING GOVERNMENT SUPPORT

This invention was supported at least in part by Ministry of Science and ICT of South Korean government for research project, the title of which is “Development of an intelligent cancer cell metastasis detection multi-modal cell imaging system for personalized cancer treatment” (Project Number: 1711110292) managed by NRF(National Research Foundation of Korea).

this invention was supported at least in part by Ministry of Science and ICT of South Korean government for research project, the title of which is “Development of multi-modal brain function sensing and control technology to maintain brain function homeostasis” (Project Number: 1711105868) managed by NRF(National Research Foundation of Korea).

this invention was supported at least in part by Ministry of Trade, Industry and Energy of South Korean government for research project, the title of which is “Development of smart monitoring system for orthopedic artificial hip implant liner” (Project Number: 1415163851) managed by KEIT (Korea Evaluation Institute of Industrial Technology).

Also, this invention was supported at least in part by Ministry of Trade, Industry and Energy of South Korean government for research project, the title of which is “Development of age-friendly wearable smart healthcare system and service for real-time quantitative monitoring of urination and defecation disorders” (Project Number: 1415173934) managed by KEIT (Korea Evaluation Institute of Industrial Technology).

### CROSS-REFERENCE TO RELATED APPLICATION

This present application claims the benefit of priority to Korean Patent Application No. 10-2020-0145463, entitled “BLADDER MONITORING APPARATUS AND METHOD FOR CONTROLLING BLADDER MONITORING APPARATUS,” filed on Nov. 3, 2020, in the Korean Intellectual Property Office, the entire disclosure of which is incorporated herein by reference.

### FIELD

The present disclosure relates to a technique of accurately determining a bladder status of a subject based on an ultrasound image and a pose of the subject.

### BACKGROUND

A bladder is a pouch-shaped organ in a lower abdomen of a human body. The bladder functions to store urine flowing from the kidney and when it is full, contract in an appropriate environment to drain the urine to the outside through urethra. The urethral sphincter contributes to storing urine and remains open during the urination. Generally, the functions of the bladder and the urethral sphincter are controlled by a nervous system, such as a brain or a spinal cord.

When a structural abnormality of the bladder or the urethra is found or an abnormality of the nervous system which controls the bladder and the urethra is found, urine storage disorder symptoms such as frequent urination, nocturia, urgency, or urge incontinence or drainage disorder symptoms such as delayed urination, weak urination, interrupted voiding, or abdominal pressure voiding may occur.

Many of patients who have a break in the nervous system connected from the brain to the bladder or the urethra due to radical surgery in the pelvic cavity, such as the uterine cancer or the rectal cancer or patients with nervous system abnormalities accompanied with paralysis due to spinal cord injury or spinal cord disease have impairments in the autonomous urine drainage control function so that it is common to always have a large amount of residual urine in the bladder. When the urine is not excreted in a timely manner, it may result in chronic urinary retention.

Specifically, persistent bladder overfilling due to urinary retention may result in frequent urinary tract infections and even kidney damages. Further, the urine overflows from the bladder so that the patient suffers the urinary incontinence, which may cause poor hygiene and serious deterioration in a quality of life. The problem of urinary incontinence causes psychological anxiety in patients and a lot of inconveniences while going out. For the patients with urine drainage disorders, periodic artificial urine drainage is essential.

Among the artificial urine drainage methods, the intermittent self-catheterization method which inserts a catheter to the bladder through the patient's urethra to intermittently drain the urine at every few hours is the easiest and least invasive method and is most commonly applied in reality. However, the urine volume varies greatly depending on a personal habit and even for the same person, there is a big change depending on a type and an amount of intake. Therefore, it is not easy to predict the fullness of the bladder in advance so that it is very hard to set the catheterization interval at a specific time. Accordingly, patients with bladder dysfunction or their families or caregivers who take care of the patients are often in a situation where they do not know when to artificially drain the urine. If the need to urinate due to the bladder fullness can be known, it would be very useful to prevent complications such as urinary incontinence, urinary tract infection, or kidney damage caused in the patients due to drainage disorders.

In the related art (Registered Patent No. 10-307085), a configuration which calculates a distance between walls of the bladder using an ultrasonic signal, and then when the distance between the walls of the bladder exceeds a predetermined distance, issues a warning is disclosed. However, since only the distance between the walls is measured, a residual urine volume cannot be exactly measured so that there is a limit to accurately judging the bladder status.

Further, a technique of calculating a bladder volume from a bladder image generated based on the ultrasonic signal and when the bladder volume reaches a threshold value, determining that the bladder status is a urination necessary state to notify the urination necessary state has been proposed. However, as the posture of the human body changes, not only an anteroposterior diameter (the maximum distance between the front and back walls inside the bladder) and a cephalocaudal diameter (the maximum distance between the upper and lower walls inside the bladder), but also a shape of the bladder is changed. Therefore, the bladder volume is calculated without considering the change of the posture and the bladder status is determined based thereon so that a reliability for determining the bladder status may be lowered. Further, the process of calculating the bladder volume by calculating the anteroposterior diameter and the cephalocaudal diameter from the bladder image is complex to consume a lot of data resources and time so that it is not appropriate to monitor the bladder in real time.

Therefore, a technique which accurately determines a bladder status with less data resources and time consumed is necessary.

### RELATED ART DOCUMENT

### Patent Document

Patent Document: Korean Registered Patent Publication No. 10-307085 (registered on Aug. 17, 2001)

### SUMMARY

An object of an exemplary embodiment of the present disclosure is to determine a bladder status of a subject based on a posture of the subject together with an ultrasonic image of the subject to accurately determine the bladder status of the subject regardless of the change of the posture of the subject.

Another object of an exemplary embodiment of the present disclosure is to easily and rapidly acquire a bladder volume ratio of a subject with respect to the ultrasonic image and the posture information of the current subject using a learning model trained with ultrasonic images labeled with the posture information and the bladder volume ratio of the subject and determine the bladder status of the subject based on the bladder volume ratio of the subject, thereby monitoring the bladder status in real time with less data resources and time consumed.

Further, another object of an exemplary embodiment of the present disclosure is to accurately determine the bladder status of the subject in a user-customized manner regardless of a variation for every user of a bladder size or a bladder shape of the subject by acquiring the bladder volume ratio of the subject by means of a learning model trained using a previously stored maximum bladder volume image of the user together with the ultrasonic image and posture information of the current subject, based on a Siamese network.

An exemplary embodiment of the present disclosure may be a bladder monitoring apparatus which rapidly and accurately determines the bladder status of the subject by applying a machine learning-based learning model to an ultrasonic image and posture information of a subject and a control method of the bladder monitoring apparatus.

An exemplary embodiment of the present disclosure may be a bladder monitoring apparatus which includes a processor, a memory configured to be operably connected to the processor and store at least one code executed by the processor, and a transceiver configured to receive a posture sensing signal obtained by sensing a posture of the subject based on a sensor. The memory stores a code which is executed by the processor to cause a processor to determine a bladder status of the subject by applying a machine learning-based learning model to an ultrasonic image generated from a reflection ultrasonic signal and posture information generated based on a posture sensing signal.

Further, an exemplary embodiment of the present disclosure may be a method for controlling a bladder monitoring apparatus implemented by the bladder monitoring apparatus including a step of allowing the bladder monitoring apparatus to receive a reflection ultrasonic signal acquired from a subject by an external device including an inertial measurement unit (IMU) sensor and an ultrasonic transducer and a posture sensing signal obtained by sensing a posture of the subject by an IMU sensor and a step of allowing the bladder monitoring apparatus to determine a bladder status of the subject by applying a machine learning-based learning model to an ultrasonic image generated from the reflection ultrasonic signal and the posture information generated based on the posture sensing signal.

Other aspects, features, and advantages other than those described above will become apparent from the following drawings, claims, and the detailed description of the present invention.

According to the exemplary embodiments of the present disclosure, a bladder status of a subject is determined based on a posture of the subject together with an ultrasonic image of the subject to accurately determine the bladder status of the subject regardless of the change of the posture of the subject.

According to the exemplary embodiments of the present disclosure, it is possible to easily and rapidly acquire a bladder volume ratio of a subject with respect to the ultrasonic image and the posture information of the current subject using a learning model trained with ultrasonic images labeled with the posture information and the bladder volume ratio of the subject and determine the bladder status of the subject based on the bladder volume ratio of the subject, thereby monitoring the bladder status in real time with less data resources and time consumed.

Further, according to the exemplary embodiments of the present disclosure, it is possible to accurately determine the bladder status of the subject in a user-customized manner regardless of a variation for every user of a bladder size or a bladder shape of the subject by acquiring the bladder volume ratio of the subject by means of a learning model trained using a previously stored maximum bladder volume image of the user together with the ultrasonic image and posture information of the current subject, based on a Siamese network.

### BRIEF DESCRIPTION OF THE DRAWINGS

The foregoing and other aspects, features, and advantages of the invention, as well as the following detailed description of the embodiments, will be better understood when read in conjunction with the accompanying drawings. For the purpose of illustrating the present disclosure, there is shown in the drawings an exemplary embodiment, it being understood, however, that the present disclosure is not intended to be limited to the details shown because various modifications and structural changes may be made therein without departing from the spirit of the present disclosure and within the scope and range of equivalents of the claims. The use of the same reference numerals or symbols in different drawings indicates similar or identical items.

FIG. 1 is a view schematically illustrating a configuration of a bladder monitoring system including a bladder monitoring apparatus, an external device, and a network connecting the bladder monitoring apparatus and the external device according to an exemplary embodiment of the present disclosure;

FIG. 2 is a view schematically illustrating another configuration of a bladder monitoring system including a bladder monitoring apparatus, an external device, a server, and a network connecting them according to an exemplary embodiment of the present disclosure;

FIG. 3 is a view illustrating an example of a configuration of an external device which communicates with a bladder monitoring apparatus according to an exemplary embodiment of the present disclosure;

FIG. 4 is a view illustrating a wearing example of an external device;

FIG. 5 is a view illustrating an example of an ultrasonic transducer in an external device;

FIG. 6 is a view illustrating an example of a configuration of a bladder monitoring apparatus according to an exemplary embodiment of the present disclosure;

FIG. 7 is a view for explaining a learning example of a learning model used for a bladder monitoring apparatus according to an exemplary embodiment of the present disclosure;

FIG. 8 is a view for explaining a concept of a method of estimating a bladder volume percentage of a subject using a learning model in a bladder monitoring apparatus according to an exemplary embodiment of the present disclosure;

FIG. 9 is a view for explaining a method of estimating a bladder volume percentage of a subject using a learning model in a bladder monitoring apparatus according to an exemplary embodiment of the present disclosure;

FIG. 10 is a view for explaining an example of estimating a user-customized bladder volume percentage in a bladder monitoring apparatus according to an exemplary embodiment of the present disclosure; and

FIG. 11 is a flowchart illustrating a method for controlling a bladder monitoring apparatus according to an exemplary embodiment of the present disclosure.

### DETAILED DESCRIPTION

Advantages and characteristics of the present disclosure and a method of achieving the advantages and characteristics will be clear by referring to exemplary embodiments described below in detail together with the accompanying drawings. However, the description of particular exemplary embodiments is not intended to limit the present disclosure to the particular exemplary embodiments disclosed herein, but on the contrary, it should be understood that the present disclosure is to cover all modifications, equivalents and alternatives falling within the spirit and scope of the present disclosure. The exemplary embodiments disclosed below are provided so that the present disclosure will be thorough and complete, and also to provide a more complete understanding of the scope of the present disclosure to those of ordinary skill in the art. In describing the present invention, when it is determined that a detailed description of related well-known technology may obscure the gist of the present invention, the detailed description thereof will be omitted.

Terms used in the present application are used only to describe specific exemplary embodiments, and are not intended to limit the present invention. A singular form may include a plural form if there is not clearly opposite meaning in the context. In the present application, it should be understood that the term “include” or “have” indicates that a feature, a number, a step, an operation, a component, a part or the combination thoseof described in the specification is present, but do not exclude a possibility of presence or addition of one or more other features, numbers, steps, operations, components, parts or combinations, in advance. Terminologies such as first or second may be used to describe various components but the components are not limited by the above terminologies. The above terms are used only to distinguish one component from the other component.

Hereinafter, exemplary embodiments according to the present invention will be described in detail with reference to the accompanying drawings. In the description with reference to the accompanying drawings, like reference numbers and designations in the various drawings indicate like elements and a redundant description thereof will be omitted.

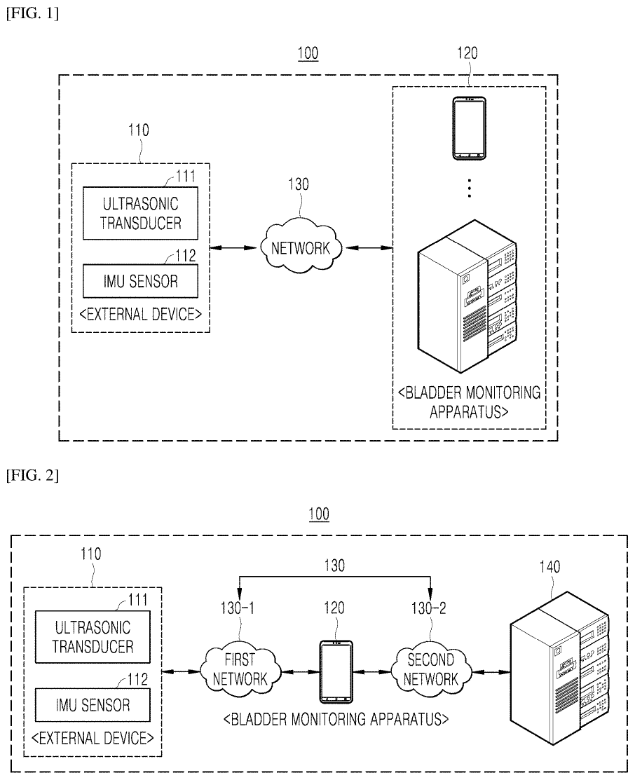

FIG. 1 is a view schematically illustrating a configuration of a bladder monitoring system including a bladder monitoring apparatus, an external device, and a network connecting the bladder monitoring apparatus and the external device according to an exemplary embodiment of the present disclosure.

Referring to FIG. 1, the bladder monitoring system 100 may include an external device 110, a bladder monitoring apparatus 120, and a network 130.

The external device 110 is a wearable device and is formed as a belt type or a patch type to be wearable on a subject (for example, a human body). The external device 110 may include an ultrasonic transducer 111 and an inertial measurement unit (IMU) sensor 112.

The ultrasonic transducer 111 may generate a transmission ultrasonic signal to the subject and acquire a reflection ultrasonic signal reflected from the subject.

The IMU sensor 112 may acquire a sensing value obtained by measuring at least one of a specific force, an angle ratio, and a magnetic field around the sensor from the subject using a combination of an accelerometer and a tachometer or a magnetometer.

The external device 110 may transmit the reflection ultrasonic signal acquired by the ultrasonic transducer 111 and the sensing value acquired by the IMU sensor 112 to the bladder monitor apparatus 120.

The bladder monitoring apparatus 120 may be, for example, a mobile terminal or a server and receive a reflection ultrasonic signal acquired by the ultrasonic transducer 111 from the external device 110. The bladder monitoring apparatus 120 may further receive a sensing value acquired by the IMU sensor 112 from the external device 110, as a posture sensing signal.

The bladder monitoring apparatus 120 may determine the bladder status of the subject based on the posture sensing signal and the reflection ultrasonic signal received from the external device 110. At this time, the bladder monitoring apparatus 120 generates posture information based on the posture sensing signal obtained by sensing a posture of the subject by the IMU sensor 112 and generates an ultrasonic image (for example, a two-dimensional ultrasonic image) from the reflection ultrasonic signal, and then determines the bladder status of the subject by applying a machine learning-based learning model to the posture information and the ultrasonic image to rapidly determine a bladder status of the subject.

Here, the posture information may be data obtained by pre-processing the posture sensing signal to be inputtable to the learning model by the bladder monitoring apparatus 120, or a posture determined as one of a plurality of postures (for example, a standing posture, a sitting posture, or a lying posture) set in advance based on the posture sensing signal.

Further, the learning model may be acquired from a database of the bladder monitoring apparatus 120 or an external server and output a bladder volume percentage of the subject as the ultrasonic image and the posture information are input. Here, the bladder volume percentage may refer to a percentage of a current bladder volume with respect to a maximum bladder volume in which the bladder is fully filled with urine.

The bladder monitoring apparatus 120 may determine whether the bladder status of the subject is a urination necessary state or not based on the output bladder volume percentage of the subject, as a result of applying the machine learning-based learning model to the posture information and the ultrasonic image. Specifically, the bladder monitoring apparatus 120 determines that the bladder status of the subject is a urination necessary state when the output bladder volume percentage of the subject exceeds the predetermined reference value, to generate a urination-indicating message. As the urination-indicating message is output, a supervisor (for example, a nurse or a caregiver) handles the urination of a user (for example, a patient) who is unable to urinate by him/herself due to dysuria at a necessary time, without delay, to prevent the user from getting a urinary tract infection or depression.

The network 130 may connect the external device 110 and the bladder monitoring apparatus 120. The network 130 may include, for example, wired networks such as local area networks (LANs), wide area networks (WANs), metropolitan area networks (MANs), and integrated service digital networks (ISDNs), or wireless networks such as wireless LANs, CDMA, Bluetooth, and satellite communication, but the scope of the present disclosure is not limited thereto. Further, the network 130 may transmit and receive information using short-range communication and/or long-range communication. Here, the short-range communications may include Bluetooth, radio frequency identification (RFID), infrared data association (IrDA), ultra-wideband (UWB), ZigBee, and wireless fidelity (Wi-Fi) technologies and the long-range communications may include code division multiple access (CDMA), frequency division multiple access (FDMA), time division multiple access (TDMA), orthogonal frequency division multiple access (OFDMA), and single carrier frequency division multiple access (SC-FDMA) technologies.

FIG. 2 is a view schematically illustrating another configuration of a bladder monitoring system including a bladder monitoring apparatus, an external device, a server, and a network connecting them according to an exemplary embodiment of the present disclosure.

Referring to FIG. 2, the bladder monitoring system 100 may include an external device 110, a bladder monitoring apparatus 120, a network 130, and a server 140. A basic configuration of the bladder monitoring system 100 is the same as the bladder monitoring system 100 described with reference to FIG. 1 so that a description thereof will be omitted.

However, as long as the bladder monitoring apparatus 120 is a computing device including a processor and a memory configured to store a code which executes the processor, such as a laptop computer or a tablet computer, a type thereof is not specifically limited and for example, the bladder monitoring apparatus 120 may be a mobile terminal. When the bladder monitoring apparatus 120 receives a reflection ultrasonic signal which is acquired by the ultrasonic transducer 111 from the external device 110, the bladder monitoring apparatus 120 may generate an ultrasonic image from the reflection ultrasonic signal.

The bladder monitoring apparatus 120 may transmit the ultrasonic image to the server 140 together with a sensing value of the IMU sensor 112 received as a posture sensing signal from the external device 110.

The server 140 which receives the posture sensing signal and the ultrasonic image from the bladder monitoring apparatus 120 may generate posture information based on the posture sensing signal and determine a bladder status of the subject based on the posture information and the reflection ultrasonic signal. At this time, the server 140 applies the machine learning-based learning model to the posture information and the ultrasonic image to determine a bladder status of the subject to quickly determine the bladder status of the subject. Here, the posture information may be data obtained by pre-processing the posture sensing signal to be inputtable to the learning model by the server 140 or a posture determined as one of a plurality of postures set in advance based on the posture sensing signal.

The network 130 may include a first network 130-1 between the external device 110 and the bladder monitoring apparatus 120 and a second network 130-2 between the bladder monitoring apparatus 120 and the server 140.

Here, the first network 130-1 may transmit and receive information using a short-range communication. Here, the short-range communication includes Bluetooth, RFID, infrared communication, UWB, ZigBee, and Wi-Fi technologies.

The second network 130-2 may transmit and receive information using long-range communication. Here, the long-range communication includes, for example, CDMA, FDMA, TDMA, OFDMA, and SC-FDMA techniques.

The server 140 determines the bladder status of the subject, instead of the bladder monitoring apparatus 120, to reduce the burden on data to be processed by the bladder monitoring apparatus 120 which is a mobile terminal to determine the bladder status of the bladder of the subject.

When the server 140 identifies that the output bladder volume percentage of the subject exceeds the predetermined reference value, as the result of applying the machine learning-based learning model to the posture information and the ultrasonic image, the server 140 determines the bladder status of the subject as a urination necessary status to generate the urination-indicating message and transmit the urination-indicating message to the bladder monitoring apparatus 120. At this time, the bladder monitoring apparatus 120 outputs the urination-indicating message received from the server 140 to notify the urination necessary time to process the urination of the user at an appropriate time.

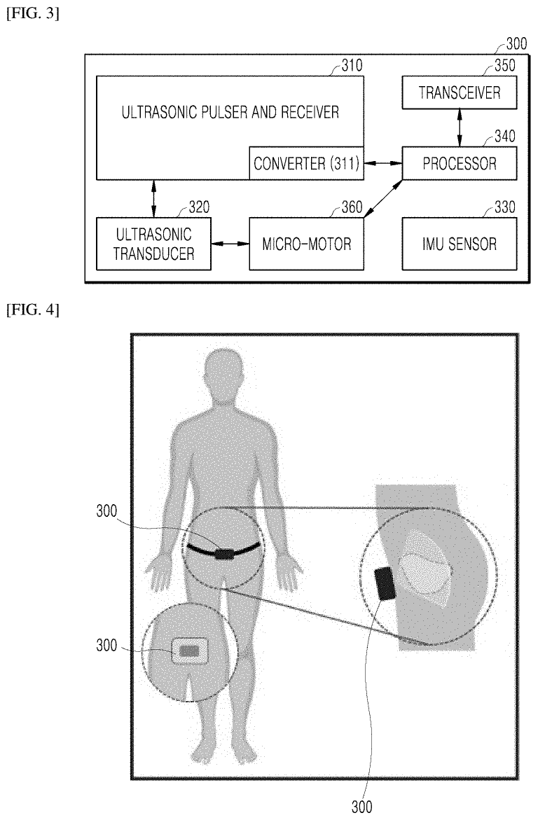

FIG. 3 is a view illustrating an example of a configuration of an external device which communicates with a bladder monitoring apparatus according to an exemplary embodiment of the present disclosure. FIG. 4 is a view illustrating a wearing example of an external device and FIG. 5 is a view illustrating an example of an ultrasonic transducer in an external device.

Referring to FIG. 3, the external device 300 may include an ultrasonic pulser and receiver 310, an ultrasonic transducer 320, an IMU sensor 330, a processor 340, and a transceiver 350. Further, the external device 300 may further include a micro-motor 360. The external device 300 is a wearable device, for example, is formed as a belt type or a patch type to be worn on the subject as illustrated in FIG. 4.

The ultrasonic pulser and receiver 310 may generate a transmission signal in response to the control of the processor 340 to transmit the transmission signal to the ultrasonic transducer 320 and generate ultrasonic data using a reception signal received from the ultrasonic transducer 320. At this time, the ultrasonic pulser and receiver 310 may convert an analog reception signal received from the ultrasonic transducer 320 into a digital signal by means of a converter 311 (an analog-digital converter) and generate ultrasonic data using the digital-converted reception signal.

The ultrasonic transducer 320 may be a single-element probe 510 or multi-element probe 520, as illustrated in FIG. 5. Further, the ultrasonic transducer 320 may be, for example, a linear type probe, but is not limited thereto and may be a convex type probe.

The ultrasonic transducer 320 may generate a transmission ultrasonic signal to the subject in accordance with the transmission signal applied from the ultrasonic pulser and receiver 310. The ultrasonic transducer 320 may receive a reflection ultrasonic signal reflected from the subject to form a reception signal and transmit the reception signal to the ultrasonic pulser and receiver 310.

The IMU sensor 330 acquires a sensing value associated with the posture in response to the control of the processor 340 to provide the sensing value to the processor 340. At this time, the IMU sensor 330 may acquire a sensing value obtained by measuring at least one of a specific force, an angle ratio, and a magnetic field around the sensor from the subject using a combination of an accelerometer and a tachometer or a magnetometer.

The processor 340 may include a field programmable gate array (FPGA) and a central processing unit (CPU) and control the ultrasonic pulser and receiver 310, the ultrasonic transducer 320, and the IMU sensor 330 and process data based on a signal input from the bladder monitoring apparatus (not illustrated) by means of the transceiver 350 to transmit the data to the bladder monitoring apparatus by means of the transceiver 350.

The transceiver 350 may be configured by a communication chip to be communicable with the bladder monitoring apparatus and support the wireless communication.

The micro-motor 360 is an ultra-small size motor. When the ultrasonic transducer 320 is a single element, the micro-motor may be used for sector-scanning.

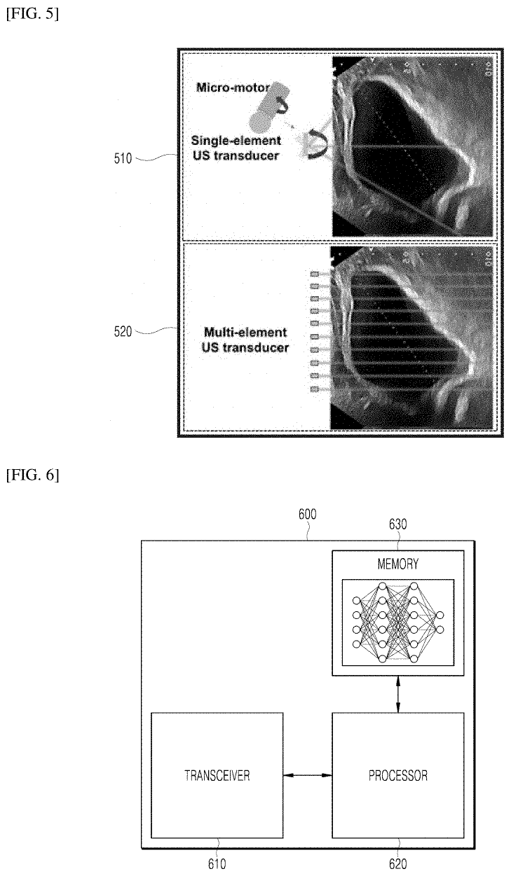

FIG. 6 is a view illustrating an example of a configuration of a bladder monitoring apparatus according to an exemplary embodiment of the present disclosure.

Referring to FIG. 6, the bladder monitoring apparatus 600 according to an exemplary embodiment of the present disclosure may include a transceiver 610, a processor 620, and a memory 630.

The transceiver 610 may receive a reflection ultrasonic signal from the subject and a posture sensing signal obtained by sensing a posture of the subject based on the sensor. Here, the posture sensing signal may be a sensing value measured from an IMU sensor of an external device worn on the subject. The external device is a wearable device and is formed as a belt type or a patch type to be worn to be in close contact with the subject regardless of a portion worn on the subject.

The processor 620 may generate the ultrasonic image from the reflection ultrasonic signal or receive an ultrasonic image generated from the reflection ultrasonic signal from a terminal (for example, a mobile terminal) by means of a transceiver 610.

Further, the processor 620 may generate posture information based on the posture sensing signal or receive posture information generated from the posture sensing signal from the terminal (for example, a mobile terminal) by means of the transceiver 610. Here, the posture information may be data obtained by pre-processing the posture sensing signal to be inputtable to the learning model or a posture determined as one of a plurality of postures set in advance based on the posture sensing signal.

The processor 620 applies the machine learning-based learning model to the ultrasonic image and the posture information to determine the bladder status of the subject. Here, the learning model is trained in advance by the processor 620 with ultrasonic images labeled with posture information and the bladder volume percentage of the subject to be stored in the memory 630 or received from the external server to be stored in the memory 630. Specifically, the processor 620 may apply the learning model of outputting a bladder volume percentage of the subject as the ultrasonic image and the posture information are input. At this time, as the processor 620 acquires the bladder volume percentage of the subject using the learning model, as compared with the bladder volume calculating process of the related art (that is, the bladder volume is calculated by calculating the anteroposterior diameter and the cephalocaudal diameter from the ultrasonic image), the bladder volume percentage of the subject may be rapidly acquired. Further, the processor 620 acquires the bladder volume percentage of the subject using the posture information together with the ultrasonic image and determines the bladder status of the subject based on the bladder volume percentage to determine the bladder status of the subject in consideration of the change of the bladder shape in accordance with the posture of the subject.

As another example, the learning model may be a model which is trained to output the bladder volume percentage with a maximum bladder volume image, an ultrasonic image, and posture information of the subject which are stored in advance, as an input, based on the Siamese network.

The processor 620 compares the bladder volume percentage of the subject output from the learning model and a predetermined reference value. When the bladder volume percentage of the subject exceeds the reference value as a comparison result, the processor may determine the bladder status of the subject as a urination necessary state and when the bladder volume percentage of the subject is equal to or lower than the reference value, the processor may determine the bladder status of the subject as a urination unnecessary state. At this time, when the bladder volume percentages of the subject which are output at different times continuously exceed the reference value, the processor 620 may determine the bladder status of the subject as a urination necessary state. By doing this, the processor 620 does not hastily determine the bladder status of the subject based on the temporary change of the bladder volume percentage of the subject, but stably determines the bladder status of the subject based on the consistent change of the bladder volume percentage of the subject so that the reliability of the determination result for the bladder status of the subject may be increased. For example, when three bladder volume percentages of the subject output as a result of applying the machine learning-based learning model to three ultrasonic images and posture information which are generated from three reflection ultrasonic signals and posture sensing signals which are continuously acquired from the subject at every five seconds exceed the reference value, the processor 620 may determine the bladder status of the subject as a urination necessary state.

According to an exemplary embodiment, the processor 620 compares the bladder volume percentage of the subject output from the learning model and a predetermined reference value for every step, and based on the result of identifying a step corresponding to the bladder volume percentage of the subject as the comparison result, may determine the bladder status of the subject as a setting state corresponding to the step (for example, a state in which the bladder is almost empty (less than 30% of the bladder volume percentage)), a state in which the bladder is half filled (30% or more and less than 60% of the bladder volume percentage), and a state in which the bladder is almost full (90% or higher of the bladder volume percentage).

The processor 620 generates urination-indicating message as the bladder status of the subject is determined as a urination necessary state to output the message to an output interface (for example, a speaker or a display, not illustrated) or transmits the message to the terminal (for example, a mobile terminal) to allow the terminal to output the urination-indicating message to allow a supervisor who checks the urination-indicating message to handle the urination of the user at a proper time. Here, the urination-indicating message may include, for example, a bladder volume percentage, a urination necessary time, a urination necessary time history, and a previous urine volume.

The processor 620 counts the number of times of determining the bladder status of the subject as a urination necessary state for a predetermined time and generates and provides at least one of the counted number and the increasing speed of the number to identify the urine volume of the user which is processed for a predetermined period and a urination cycle change.

Further, the processor 620 may generate a catheter control signal based on the bladder status of the subject. At this time, the processor 620 may generate a catheter opening signal based on the determination that the bladder status of the subject is a urination necessary state to transmit the catheter opening signal to an automatic openable catheter (not illustrated) connected to the bladder of the subject so that the urination may be automatically processed without having intervention of the supervisor.

According to an exemplary embodiment, the processor 620 determines whether an activity of the subject is normal based on a posture sensing signal received at every predetermined cycle by means of the transceiver 610, and when it is determined that the activity of the subject is abnormal, the processor generates and outputs a warning message or transmits the warning message to the terminal (for example, a mobile terminal) to allow the terminal to output the warning message. The supervisor who checks the warning message quickly checks whether the user is not normal to immediately identify the problem of the user or solve the inconvenience of the user.

The memory 630 may be operably connected to the processor 620 and store at least one code in association with an operation performed in the processor 620. Further, the memory 630 may further store a learning model which outputs the bladder volume percentage of the subject.

Further, the memory 630 may perform a function of temporarily or permanently storing data processed by the processor 620. Here, the memory 630 may include magnetic storage media or flash storage media, but the scope of the present disclosure is not limited thereto. The memory 630 may include an embedded memory and/or an external memory and also include a volatile memory such as a DRAM, an SRAM, or an SDRAM, a non-volatile memory such as an one time programmable ROM (OTPROM), a PROM, an EPROM, an EEPROM, a mask ROM, a flash ROM, an NAND flash memory, or an NOR flash memory, a flash drive such as an SSD, a compact flash (CF) card, an SD card, a micro-SD card, a mini-SD card, an Xd card, or a memory stick, or a storage device such as an HDD.

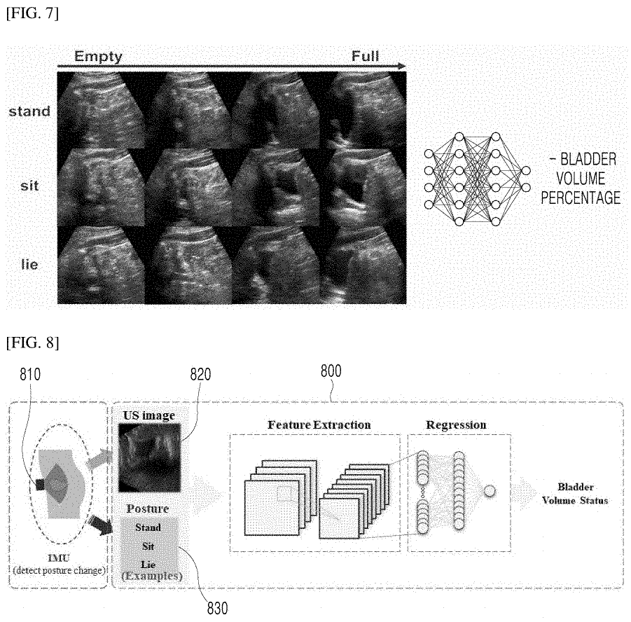

FIG. 7 is a view for explaining a learning example of a learning model used for a bladder monitoring apparatus according to an exemplary embodiment of the present disclosure.

Referring to FIG. 7, the learning model may be trained by the bladder monitoring apparatus (or a server) with ultrasonic images labeled with posture information (for example, a standing posture, a sitting posture, or a lying posture) of the subject and the bladder volume percentage.

The ultrasonic images which are training data for training the learning model may be images acquired at every cycle set for every posture of the subject from a minimum bladder status in which the bladder of the subject is empty to a maximum bladder status in which the bladder is fully filled with urine.

At this time, the ultrasonic image in the maximum bladder status of the subject may be labeled with 100% of bladder volume percentage. Further, another ultrasonic image may be labeled with a bladder volume percentage calculated based on a result of comparing the size of the bladder, with respect to the ultrasonic image when the bladder of the subject has a maximum volume (that is, a maximum bladder volume image). As another example, another ultrasonic image may be training data labeled with a bladder volume percentage calculated based on the comparison result of image acquiring times, with respect to the time when the ultrasonic image is acquired in the maximum bladder status of the subject.

For example, among 10 ultrasonic images acquired at a predetermined cycle while changing from a minimum state in which the bladder of the subject is empty to a maximum state in which the bladder is fully filled with urine, an ultrasonic image in the maximum state is labeled with “100%” of bladder volume percentage and the ultrasonic image in an empty state is labeled with “0%” of bladder volume percentage. Another ultrasonic image may be training data labeled with bladder volume percentages which are sequentially increased by “10%”.

Further, the plurality of ultrasonic images for the bladder of the subject acquired for every posture of the subject may be training data labeled with the corresponding posture information together with the bladder volume percentage of each ultrasonic image.

The bladder monitoring apparatus (or the server) may train the learning model by means of the machine learning to output the bladder volume percentage of the subject, for each of the plurality of ultrasonic images for the subject, based on the training data labeled with the bladder volume percentage and the posture information. At this time, the bladder monitoring apparatus (or the server) uses not only the bladder volume percentage, but also the plurality of ultrasonic images labeled with the posture information as training data to train the learning model to accurately estimate and output the bladder volume percentage regardless of the bladder shape which changes according to the posture. The learning model may be a learning model based on regression analysis to estimate the bladder volume percentage which is a consecutive value.

According to another exemplary embodiment, the learning model includes two CNNs and may include a Siamese network trained to input a maximum bladder ultrasonic image and a normal bladder ultrasonic image to each CNN to make a distance between encoded result values which are outputs of the networks proportional to the difference of the bladder areas in two images. Alternatively, two ultrasonic images are input to the CNNs so that a concatenated vector to which an encoded result value which is an output of each network is connected may be connected to a fully connected layer and a final output layer may be trained to output an estimated bladder volume percentage in the normal bladder ultrasonic image.

When the learning model including the Siamese network is trained, the learning model inputs two images, that is, the maximum bladder ultrasonic image and the normal bladder ultrasonic image, for the same bladder, to two CNNs. In this case, the learning model may receive a normal bladder ultrasonic image having different bladder statuses according to the change in the urine volume, together with the maximum bladder ultrasonic image. The learning model may be trained to output a bladder volume percentage labeled to the ultrasonic image (for example, the maximum bladder ultrasonic image and the normal bladder ultrasonic image), based on the feature difference between two images. Further, when the ultrasonic image to which the posture information is further labeled, together with the bladder volume percentage, the learning model including Siamese network may be trained to output the bladder volume percentage based on the feature difference between two images and the posture information.

For example, the learning model including the Siamese network may be trained to input a maximum bladder ultrasonic image for a bladder “A” and an ultrasonic image labeled with “10%” of bladder volume percentage and the “standing posture” to two CNNs to output “10%” of bladder volume percentage and input a maximum bladder ultrasonic image for the bladder “A” and an ultrasonic image labeled with “20%” of bladder volume percentage and the “standing posture” to output “20%” of bladder volume percentage. At this time, the maximum bladder ultrasonic image may also be an image acquired in the state of “standing posture”.

Further, the learning model including the Siamese network may be trained to input a maximum bladder ultrasonic image for a bladder “B” and an ultrasonic image labeled with “10%” of bladder volume percentage and the “sitting posture” to two CNNs to output “10%” of bladder volume percentage and input a maximum bladder ultrasonic image for the bladder “B” and an ultrasonic image labeled with “20%” of bladder volume percentage and the “sitting posture” to output “20%” of bladder volume percentage.

As described above, the learning model including the Siamese network learns the maximum bladder volume image for the same bladder and an ultrasonic image of different bladder status (or different postures) according to the change in the urine volume as training data to prepare an environment in which the bladder volume percentage is estimated regardless of body conditions, ages, and genders of various users.

FIG. 8 is a view for explaining a concept of a method of estimating a bladder volume percentage of a subject using a learning model in a bladder monitoring apparatus according to an exemplary embodiment of the present disclosure.

Referring to FIG. 8, the bladder monitoring apparatus 800 receives a reflection ultrasonic signal and a posture sensing signal from an external device 810 worn on the subject and applies a machine learning-based learning model to an ultrasonic image 820 generated from the reflection ultrasonic signal and posture information 830 (for example, a standing posture, a sitting posture, and a lying posture) generated based on the posture sensing signal to determine the bladder status of the subject. The learning model may extract a feature using a convolutional neural network (CNN) and perform the regression analysis using a fully connected layer (FC) on the extracted features to estimate a bladder volume percentage (that is, a ratio of a current bladder volume with respect to a maximum bladder volume, or a quantitative bladder volume value percentage) and output the estimated bladder volume percentage. For example, the regression analysis may be performed by a support vector machine (SVM).

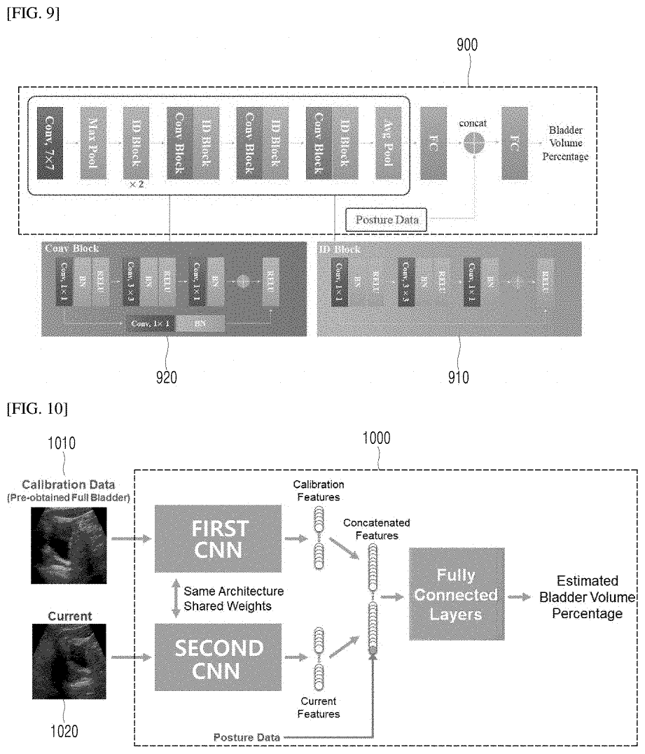

FIG. 9 is a view for explaining a method of estimating a bladder volume percentage of a subject using a learning model in a bladder monitoring apparatus according to an exemplary embodiment of the present disclosure.

Referring to FIG. 9, the bladder monitoring apparatus 900 may apply the machine learning-based learning model to the ultrasonic image and the posture information of the subject to determine the bladder status of the subject.

At this time, during the learning model applying process, the bladder monitoring apparatus 900 may apply Conv 7×7, Conv Block, Pool (Max Pool, Avg Pool), ID Block, FC and estimate and output the bladder volume percentage of the subject based on the posture data of the subject.

Here, the ID block 910 may be configured by a combination of Conv 1×1, BN (batch normalization), RELU (rectified linear unit), and Conv 3×3.

Similarly, to the ID block 910, the Conv block 920 may be configured by the Conv 1×1, BN, RELU, Conv 3×3 and further include Conv 1×1 and BN.

FIG. 10 is a view for explaining an example of estimating a user-customized bladder volume percentage in a bladder monitoring apparatus according to an exemplary embodiment of the present disclosure.

Referring to FIG. 10, a bladder monitoring apparatus 1000 estimates a bladder volume percentage for the current bladder of the user based on the maximum bladder volume of the user and determines the bladder status of the user based on the estimated bladder volume percentage so that even though the volume of the bladder varies depending on the body condition, the age, and the gender of the user, a user-customized bladder volume percentage is estimated without being affected thereby to accurately determine the bladder status of the user.

To this end, before monitoring the bladder status of the user, as the bladder of the user is fully filled with urine, the bladder monitoring apparatus 1000 may acquire an ultrasonic image when the bladder volume is maximum, that is, a maximum bladder volume image 1010, in advance, by a commercial device (for example, a commercial ultrasonic bladder scanner or a commercial ultrasonic device) to store the ultrasonic image or receive the ultrasonic image from the server.

Thereafter, the bladder monitoring apparatus 1000 inputs the maximum bladder volume image of the user and the reflection ultrasonic signal-based ultrasonic image received from the external device to the machine learning-based learning model (algorithm) including a previously trained Siamese network to estimate the bladder volume percentage. In this case, the posture sensing signal-based posture information may be further input to the learning model. The bladder monitoring apparatus 1000 applies the maximum bladder volume image of the user to the learning model together with the reflection ultrasonic signal-based ultrasonic image (or posture sensing signal-based posture information) received from the external device, that is, a current ultrasonic image (present posture information) for the bladder of the user to estimate the bladder volume percentage of the subject to accurately estimate the current bladder volume percentage of the user. Here, the learning model may be a model which is trained to output the bladder volume percentage with a maximum bladder volume image, an ultrasonic image, and posture information of the subject which are stored in advance, as an input, based on the Siamese network.

At this time, the bladder monitoring apparatus 1000 extracts a maximum feature for the maximum bladder volume image 1010 using a first CNN and extracts a current feature for the current ultrasonic image 1020 of the user using a second CNN and one vector in which the vector of the maximum feature and the vector of the current feature are concatenated is connected to a fully connected layer to estimate the bladder volume percentage of the user based on the regression analysis. The first CNN and the second CNN are Siamese networks having the same parameter.

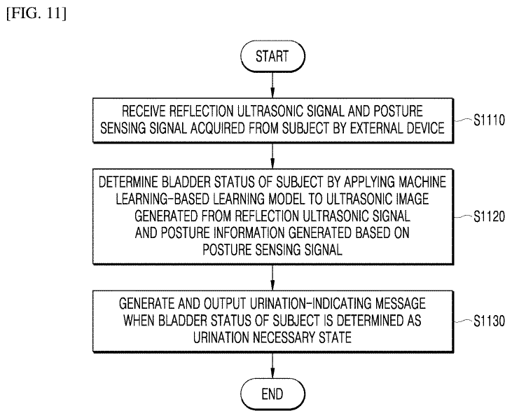

FIG. 11 is a flowchart illustrating a method for controlling a bladder monitoring apparatus according to an exemplary embodiment of the present disclosure. Here, the method for controlling the bladder monitoring apparatus may be implemented by the bladder monitoring apparatus.

Referring to FIG. 11, in step S1110, the bladder monitoring apparatus may receive a reflection ultrasonic signal acquired from the subject by an external device and a posture sensing signal obtained by sensing a posture of the subject by an IMU sensor. Here, the external device may include an IMU sensor and an ultrasonic transducer. The external device is a wearable device and is formed as a belt type or a patch type to be worn to be in close contact with the subject regardless of a portion worn on the subject.

The bladder monitoring apparatus may generate the ultrasonic image from the reflection ultrasonic signal or receive the ultrasonic image generated from the reflection ultrasonic signal from the terminal (for example, a mobile terminal).

Further, the bladder monitoring apparatus may generate posture information based on the posture sensing signal or receive posture information generated from the posture sensing signal from the terminal (for example, a mobile terminal). Here, the posture information may be data which is preprocessed the posture sensing signal to be inputtable to the learning model or a posture determined as one of a plurality of postures (for example, a standing posture, a sitting posture, or a lying posture) set in advance based on the posture sensing signal.

In step S1120, the bladder monitoring apparatus applies the machine learning-based learning model to the ultrasonic image generated from the reflection ultrasonic signal and the posture information generated based on the posture sensing signal to determine the bladder status of the subject. Here, the learning model may be trained in advance with the ultrasonic images labeled with the posture information and the bladder volume percentage of the subject.

Specifically, when the ultrasonic image and the posture information are input, the bladder monitoring apparatus applies the learning model which outputs the bladder volume percentage of the subject and when the output bladder volume percentage of the subject exceeds a predetermined reference value, may determine the bladder status of the subject as a urination necessary state. The bladder monitoring apparatus acquires the bladder volume percentage of the subject using the posture information together with the ultrasonic image and determines the bladder status of the subject based on the bladder volume percentage to determine the bladder status of the subject in consideration of the change of the bladder shape in accordance with the posture of the subject.

According to an exemplary embodiment, when the bladder volume percentages of the subject which are output at different times continuously exceed the reference value, the bladder monitoring apparatus may determine the bladder status of the subject as a urination necessary state so that when the bladder volume percentage of the subject consistently exceeds the reference value, the bladder monitoring apparatus may accurately determine the bladder status of the subject as a urination necessary state.

As another example, the learning model may be a model which is trained to output the bladder volume percentage with a maximum bladder volume image, an ultrasonic image, and posture information of the subject which are stored in advance, as an input, based on the Siamese network.

In step S1130, when the bladder status of the subject is determined as a urination necessary state, the bladder monitoring apparatus may generate and output a urination-indicating message. At this time, the bladder monitoring apparatus outputs the message through the output interface (for example, a speaker or a display) or transmits the message to the terminal (for example, a mobile terminal) to allow the terminal to output the urination-indicating message so that the supervisor who checks the urination-indicating message may handle the urination of the user at an appropriate time.

The bladder monitoring apparatus counts the number of times of determining the bladder status of the subject as a urination necessary state for a predetermined time and generates and provides at least one of the counted number and the increasing speed of the number to identify, by the supervisor, the urine volume of the user which is processed for a predetermined period and a urination cycle change to easily monitor the change of the bladder status of the user.

In the specification (specifically, claims) of the present disclosure, the terminology “said” or a similar terminology may correspond to both the singular form and the plural form. In addition, when a range is described in the present disclosure, individual values constituting the range are described in the detailed description of the present invention as including the invention to which the individual values within the range are applied (unless the context clearly indicates otherwise).

The method steps, processes, and operations described herein are not to be construed as necessarily requiring their performance in the particular order discussed or illustrated, unless specifically identified as an order of performance It is also to be understood that additional or alternative steps may be employed. It will of course be realized that while the foregoing has been given by way of illustrative example of this disclosure, all such and other modifications and variations thereto as would be apparent to those skilled in the art are deemed to fall within the broad scope and ambit of this disclosure as is herein set forth.

While the invention has been explained in relation to its embodiments, it is to be understood that various modifications thereof will become apparent to those skilled in the art upon reading the specification. Therefore, it is to be understood that the invention disclosed herein is intended to cover such modifications as fall within the scope of the appended claims.

The present disclosure described as above is not limited by the aspects described herein and accompanying drawings. It should be apparent to those skilled in the art that various substitutions, changes and modifications which are not exemplified herein but are still within the spirit and scope of the present disclosure may be made. Therefore, the scope of the present disclosure is defined not by the detailed description, but by the claims and their equivalents, and all variations within the scope of the claims and their equivalents are to be construed as being included in the present disclosure.

What is claimed is:

1. A bladder monitoring apparatus, comprising:
a processor;
a memory configured to be operably connected to the processor and store at least one code executed by the processor; and
a transceiver configured to receive a reflection ultrasonic signal from a subject and a posture sensing signal obtained by sensing a posture of the subject based on a sensor,
wherein the memory stores a code which is executed by the processor to cause the processor to apply a machine learning-based learning model to an ultrasonic image generated from the reflection ultrasonic signal and posture information generated based on the posture sensing signal to determine a bladder status of the subject.

2. The bladder monitoring apparatus according to claim 1, wherein the posture information is data obtained by pre-processing the posture sensing signal to be inputtable to the learning model or data representing a posture determined as one of a plurality of postures set in advance based on the posture sensing signal.

3. The bladder monitoring apparatus according to claim 1, wherein the learning model is trained with ultrasonic images labeled with the posture information and a bladder volume ratio of the subject.

4. The bladder monitoring apparatus according to claim 1, wherein the memory further stores a code which causes the processor to apply the learning model which outputs the bladder volume ratio of the subject as the ultrasonic image and the posture information are input.

5. The bladder monitoring apparatus according to claim 4, wherein the learning model is a model which is trained to output the bladder volume ratio with a maximum bladder volume image, the ultrasonic image, and the posture information of the subject which are stored in advance, as an input, based on Siamese network.

6. The bladder monitoring apparatus according to claim 4, wherein the memory further stores a code which causes the processor to determine the bladder status of the subject as a urination necessary state when the output bladder volume ratio of the subject exceeds a predetermined reference value.

7. The bladder monitoring apparatus according to claim 6, wherein the memory further stores a code which causes the processor to determine the bladder status of the subject as a urination necessary state when the bladder volume ratio of the subject which are output at different times continuously exceed the predetermined reference value.

8. The bladder monitoring apparatus according to claim 1, wherein the memory further stores a code which causes the processor to generate a urination-indicating message when the bladder status of the subject is determined as a urination necessary state.

9. The bladder monitoring apparatus according to claim 1, wherein the memory further stores a code which causes the processor to count the number of times of determining the bladder status of the subject as a urination necessary state for a predetermined time and generate at least one of the counted number and the increasing speed of the number.

10. The bladder monitoring apparatus according to claim 1, wherein the posture sensing signal is a sensing value measured from an inertial measurement unit (IMU) sensor of an external device worn on the subject.

11. The bladder monitoring apparatus according to claim 10, wherein the external device is formed as a belt type or a patch type.

12. The bladder monitoring apparatus according to claim 1, wherein the memory further stores a code which causes the processor to generate a catheter control signal based on the bladder status of the subject.

13. The bladder monitoring apparatus according to claim 1, wherein the transceiver receives the posture sensing signal from the subject at every predetermined cycle and the memory further stores a code which causes the processor to determine whether an activity of the subject is normal based on the posture sensing signal received at every cycle and generate a warning message when the activity of the subject is determined to be abnormal.

14. A method for controlling a bladder monitoring apparatus, the method comprising:
Receiving, by a transceiver, a reflection ultrasonic signal acquired from a subject by an external device including an inertial measurement units (IMU) sensor and an ultrasonic transducer and a posture sensing signal obtained by sensing a posture of the subject by the IMU sensor, and
Determining, by a processor, the bladder status of the subject by applying a machine learning-based learning model to an ultrasonic image generated from the reflection ultrasonic signal and posture information generated based on the posture sensing signal.

15. The method for controlling a bladder monitoring apparatus according to claim 14, wherein the determining of the bladder status of the subject includes:
Applying, by the processor, the learning model which outputs a bladder volume ratio of the subject as the ultrasonic image and the posture information are input; and
determining, by the processor, the bladder status of the subject as a urination necessary state when the output bladder volume ratio of the subject exceeds a predetermined reference value.
